(19) United States
(12) Patent Application Publication
(10) Pub. No.: US 2006/0193803 A1
(43) Pub. Date: Aug. 31, 2006

(54) Polymer particle dispersions, cosmetic compositions comprising at least one polymer particle dispersion, and cosmetic process using same

(76) Inventor: Celine Farcet, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/347,251

(22) Filed: Feb. 6, 2006

Related U.S. Application Data
(60) Provisional application No. 60/651,218, filed on Feb. 10, 2005.

(30) Foreign Application Priority Data
Feb. 4, 2005 (FR) 05 50340

(51) Int. Cl. A61K 8/00

(52) U.S. Cl. 424/70.1

(57) ABSTRACT

Disclosed herein is a dispersion of polymer particles in a liquid carbon-based medium, wherein said polymer is chosen from at least one block copolymer comprising at least one first block that is soluble in said liquid carbon-based medium and at least one second block that is insoluble in said liquid carbon-based medium. Also disclosed herein is a cosmetic composition comprising, in a cosmetically acceptable medium, said dispersion, and further disclosed is a cosmetic process for making up, cleansing, protecting against the sun, shaping, dyeing or caring for keratin materials, for example, body or facial skin, the nails, the hair and/or the eyelashes, using this composition.

This application claims benefit of U.S. Provisional Application No. 60/651,218, filed Feb. 10, 2005, the contents of which are incorporated herein by reference. This application also claims benefit of priority under 35 U.S.C. §119 to French Patent Application No. 05 50340, filed Feb. 4, 2005, the contents of which are also incorporated herein by reference.

The present disclosure relates to novel polymer particle dispersions and to their use in cosmetics; the disclosure also relates to compositions, for example, cosmetic compositions, comprising these dispersions.

It is known practice to use in cosmetics dispersions of polymer particles, generally of nanometric size, in organic media.

Thus, European Patent Application No. EP-A-0 749 747 describes a cosmetic composition comprising a polymer particle dispersion, in a non-aqueous medium, said dispersion being stabilized by adding stabilizing polymers, which bind non-covalently by means of physical interactions on the polymer particles. However, this type of composition has the following drawbacks: it requires the addition to the non-aqueous medium of a larger amount of stabilizing polymers than that effectively bound to the insoluble polymer particles, in order to obtain a relatively stable dispersion of said particles. Now, during the addition of adjuvants such as pigments to the compositions, some of the stabilizing polymers have a tendency to become desorbed from the non-soluble polymer particles and to combine with said adjuvants, which contributes towards destabilizing the dispersion, for example, by formation of aggregates from the polymer particles.

Cosmetic compositions comprising dispersions, in a non-silicone non-aqueous organic medium, of acrylic polymer particles comprising a skeleton that is insoluble in said medium, and a part that is soluble in said medium, consisting of side chains covalently bonded to said skeleton, are also known, from European Patent Application No. EP 1 428 844. In this case, the polymer particles are stabilized with a polymer (macromer) that is chemically bonded to the polymer particles.

In both cases, the nature of the stabilizing polymer is not very flexible, whether in terms of chemical nature, molar mass and/or architecture, and requires a specific synthesis. Moreover, it is not easy to modify the properties of the particle core, whether in terms of molecular mass and/or architecture.

Therefore, there is a need in the art for novel polymer particle dispersions which may afford good cosmetic properties such as good adhesion to the support (for example, skin or hair) and thus good staying power of the cosmetic composition.

Thus, the inventor has discovered, surprisingly, novel polymer particle dispersions which may afford good cosmetic properties as discussed above, and moreover, which do not comprise any stabilizer within the meaning of certain art, and thus may be much more stable over time than the usual dispersions, which implies better stability of the composition comprising it and easy formulation. In addition, the comfort of the cosmetic composition may be improved by the novel dispersions disclosed herein.

Accordingly, disclosed herein is a cosmetic composition comprising, in a cosmetically acceptable medium, at least one dispersion of polymer particles in a liquid carbon-based medium, wherein said polymer is chosen from at least one block copolymer comprising at least one first block that is soluble in said liquid carbon-based medium and at least one second block that is insoluble in said liquid carbon-based medium.

By means of the inventor's discovery disclosed herein, it is possible to modify the physicochemical properties of the dispersion, and thus of the composition comprising it, by appropriately selecting the monomers and the organic medium of which it is composed; this possibility was not available previously. This makes it possible, for example, to obtain a dispersion that has great affinity for the oily media usually used in cosmetics; this also makes it possible to prepare a dispersion whose deposit does not have any tacky nature.

Micelles of block copolymer, for example, in organic medium, are known in general. Mention may be made, for example, of PCT Patent Application No. WO 01/77198 relating to a process for preparing microgels by RAFT polymerization in the presence of a chain-transfer agent, which consists in preparing a block copolymer comprising solvophobic monomers and solvophilic monomers, and then in dispersing said block copolymer in a dispersion medium to form micelles, which will be stabilized to give the expected microgel. In said document, the dispersion medium may be organic, aqueous or aqueous-organic.

However, it is not envisaged in said document to use per se the micelles thus prepared; they serve to form microgels by crosslinking, said microgels allowing pigments or dyes to be encapsulated, for example, and being able to be used in various fields such as industrial coating. The crosslinked microgels thus obtained do not make it possible to obtain deposits that are for instance, film-forming, with good staying power and that are easy to remove;

In contrast, forming deposits that are film-forming, with good staying power and that are easy to remove is one of the benefits of the present disclosure. The polymer particle dispersion according to the disclosure thus comprises a copolymer that comprises at least one first block that is soluble in said liquid carbon-based medium and at least one second block that is insoluble in said liquid carbon-based medium.

As used herein, the term “block” is understood to mean a polymer sequence formed from several monomers, for example, from at least 5 monomers, which may be identical or different, and which may thus be in the form of a random, alternating, gradient or block, for example, diblock, triblock or multiblock, homopolymer or copolymer.

For example, the block may be of homopolymer or gradient type.

For each block, the choice of monomers and of their amount, and also of the architecture of the block, may be made by a person skilled in the art on the basis of his general knowledge so as finally to obtain a block having the required solubility (soluble or insoluble) in the liquid carbon-based medium under consideration.

The copolymer finally obtained can be, for example, of the “diblock” type, i.e. it comprises only two blocks, one being soluble in the medium, the other being insoluble; however, it may be of the “triblock” or “multiblock” (more than three blocks) type.

For example, the sequence of the soluble and insoluble blocks can be alternating. Each soluble block may be of identical or different length and/or molar mass, of identical or different chemical nature, and of identical or different architecture. Each insoluble block may be of identical or different length or molar mass, of identical or different chemical nature, and of identical or different architecture.

For example, in one embodiment, the copolymer according to the disclosure is linear; however, it may be branched and/or grafted in other embodiments.

The copolymer according to the disclosure is not crosslinked; this means that there is no deliberate addition of compound whose purpose is to crosslink (crosslinking agent).

As used herein, the term “soluble” is understood to mean that the block is fully dissolved (without any apparent deposit, or insoluble aggregate or sediment), visually, at 20° C., at a concentration of greater than or equal to 5% by weight, in the liquid carbon-based medium under consideration.

The dispersions according to the disclosure may, for example, be in the form of polymer micelles (or particles) as a stable dispersion in the medium under consideration. These micelles (or particles) can, for example, range from 5 to 1000 nm, for example, from 10 to 500 nm, further, for example, from 20 to 300 nm or even further, for example, from 30 to 200 nm in size, which can allow great stability of the dispersion over time to be obtained.

As used herein, the term “polymer micelles” is understood to mean self-dispersed particles obtained by self-assembly of the copolymers as defined below.

Thus, it may be considered that the polymerization of the monomer(s) of which the first block was composed, of initiator and/or of control agent leads to a first block that is soluble in the medium under consideration. The addition of the monomer(s) intended to compose the core of the particle leads to the formation of the copolymer, which is generally a block copolymer, of soluble and/or insoluble type, this copolymer becoming spontaneously organized into a polymer micelle, i.e. forming a self-dispersed polymer particle in the liquid carbon-based medium.

The dispersion disclosed herein may be formed, for example, in a single step dispersed copolymer particles, of which the characteristics of the soluble part and those of the core of the particle are simultaneously controllable.

The copolymers according to the present disclosure, for example, can have a number-average molecular weight (Mn) ranging from 1,000 to 700,000, for example, from 10,000 to 500,000 and further, for example, from 15,000 to 350,000, or even further, for example, from 25,000 to 150,000.

For example, the copolymer according to the disclosure can have a mass polydispersity index (Ip) of less than or equal to 6, for example, from 1.05 to 4 and further, for example, from 1.1 to 3, and even further, for example, from 1.15 to 2.5.

The mass polydispersity index (Ip) of the copolymer is equal to the ratio of the weight-average molecular mass (Mw) to the number-average molecular mass (Mn). A low mass polydispersity reflects approximately identical chain lengths.

The weight-average (Mw) and number-average (Mn) molecular masses are determined by gel permeation liquid chromatography (GPC), eluted with THF, on a calibration curve established with linear polystyrene standards, using a refractometric detector.

For example, the dispersion according to the disclosure can have a uniform particle size polydispersity, which means that all the particles are of the same size. In one embodiment, the dispersion is, for example, such that at least 50% in numerical terms of the particles of the dispersion have an identical or virtually identical diameter (difference of less than 10%); this contributes towards better stability of the dispersion over time (no decantation, flocculation and/or sedimentation).

In certain known dispersions prepared according to “conventional” free-radical polymerization processes, the mixture obtained has a heterogeneous chemical composition since it is generally a mixture of homopolymers and copolymers. This is not present in the dispersion disclosed herein.

In the present disclosure, the vast majority or even, in certain embodiments, all of the chains (depending on the chosen polymerization technique) may be in the form of copolymer, which will have the beneficial feature of improving the stability of the dispersions. Moreover, the copolymers according to the disclosure can have narrow molar mass and chemical composition distributions and controlled molar masses, which makes it possible to control the size of the particles and their size distribution.

The copolymer according to the disclosure thus comprises a first block that is soluble in the liquid carbon-based dispersion medium and at least one second block that is insoluble in said medium.

The soluble block, for example, comprises 50% to 100% by weight of monomer(s) that is(are) soluble in said medium, for example, from 60% to 90% by weight and further, for example, from 70% to 80% by weight of soluble monomer(s), alone or as a mixture. However, it may also comprise from 0% to 50% by weight, for example, from 10% to 40% by weight or further, for example, from 20% to 30% by weight of monomer(s) that is(are) insoluble in said medium, alone or as a mixture.

Similarly, the insoluble block, for example, comprises 50% to 100% by weight of monomer(s) that is(are) insoluble in said medium, for example, from 60% to 90% by weight and further, for example, from 70% to 80% by weight of insoluble monomer(s), alone or as a mixture. However, it may also comprise 0% to 50% by weight, for example, from 10% to 40% by weight and further, for example, from 20% to 30% by weight of monomer(s) that is(are) soluble in said medium, alone or as a mixture.

A person skilled in the art will know how to select, on the basis of his general knowledge, the soluble and insoluble monomer(s), and also the amounts thereof, in order finally to obtain a block having the disclosed solubility (soluble or insoluble) in the liquid carbon-based medium under consideration.

As used herein, the term “monomer that is soluble in the medium” is understood to mean any monomer whose homopolymer is in soluble form, i.e. fully dissolved at a concentration of greater than or equal to 5% by weight at room temperature (20° C.), in said medium.

As used herein, the term “insoluble monomer” is thus understood to mean any monomer whose homopolymer is not in soluble form, i.e. not fully dissolved at a concentration of greater than or equal to 5% by weight at room temperature (20° C.), in said medium. However, the insoluble monomers may, as monomers, be soluble in the medium under consideration, given that they become insoluble after polymerization.

In the dispersion disclosed herein, the proportion of soluble block and of insoluble block in the copolymer should be such that the copolymer can form a polymer micelle.

For example, the at least one insoluble block may be present in an amount ranging from 30% to 97% by weight, for example, from 40% to 95% by weight or further, for example, from 50% to 93% by weight, even further, for example, 60% to 92% by weight and even further, for example, from 75% to 90% by weight, relative to the total weight of the copolymer.

The at least one soluble block thus, for example, may be present in an amount ranging from 3% to 70% by weight, for example, from 5% to 60% by weight, further, for example, from 7% to 50% by weight, even further, for example, 8% to 40% by weight and even further, for example, from 10% to 25% by weight, relative to the total weight of the copolymer.

Among the soluble monomers that may be used, non-limiting mention may be made, alone or as a mixture, of the following monomers:
the methacrylates of formula CH₂═C(CH₃)—COOR₁
wherein R₁ is chosen from linear and branched C8-C22 alkyl groups such as lauryl, behenyl and stearyl; cyclic alkyl groups comprising 8 to 30 carbon atoms, such as isobornyl; and tert-butyl groups;
the acrylates of formula CH₂═CH—COOR₂
wherein R₂ is chosen from linear and branched C8-C22 alkyl groups such as lauryl, behenyl, stearyl or 2-ethylhexyl; cyclic alkyl groups comprising 8 to 30 carbon atoms, such as isobornyl; and isobutyl groups;
the (meth)acrylamides of formula CH₂═C(CH₃)—CONR₃R₄ or CH₂═CH—CONR₃R₄,
wherein R₃ is chosen from a hydrogen atom and linear and branched C1-C12 alkyl groups and R₄ is chosen from linear and branched C₈ to C₁₂ alkyl groups, such as isooctyl, isononyl and undecyl groups;
the di-n-alkylitaconates of formula CH₂═C(CH₂—COO(CH₂)ₙ₋₁—CH₃)—COO(CH₂)ₙ₋₁—CH₃,
wherein n is chosen from an integer greater than or equal to 5, for example, an integer ranging from 5 to 12;
the vinyl esters of formula R₅—CO—O—CH═CH₂ wherein R₅ is chosen from linear and branched C₈ to C₂₂ alkyl groups;
the ethers of vinyl alcohol and of an alcohol of formula R₆O—CH═CH₂ wherein R₆ is chosen from linear and branched alkyl groups comprising from 8 to 22 carbon atoms;
ethylenic monomers wherein the ester group contains silanes or siloxanes, and which contain only one silicon atom, such as (meth)acryloxypropyltrimethoxysilane;
carbon-based macromonomers with a polymerizable end group. As used herein, the term “macromonomer with a polymerizable end group” is understood to mean any oligomer comprising on only one of its ends a polymerizable end group capable of reacting during the polymerization reaction with ethylenic monomers. The polymerizable group of the macromonomer may, for example, be an ethylenically unsaturated group capable of undergoing free-radical polymerization. The said polymerizable end group may be, for example, a vinyl or (meth)acrylate (or (meth)acryloxy) group and, for example, a (meth)acrylate group. As used herein, the term “carbon-based macromonomer” is understood to mean a non-silicone macromonomer, and, for example, an oligomeric macromonomer obtained by polymerization of non-silicone ethylenically unsaturated monomer(s), and mainly by polymerization of acrylic monomers and/or non-acrylic vinyl monomers.

Among the carbon-based macromonomers bearing a polymerizable end group that may be used, non-limiting mention may be made of:

(i) linear and branched C6-C22 and, for example, C8-C18 alkyl(meth)acrylate homopolymers and copolymers, comprising a polymerizable end group chosen from vinyl and (meth)acrylate groups, among which further non-limiting mention may be made of: poly(2-ethylhexyl acrylate)macromonomers comprising mono(meth)acrylate end groups; poly(dodecyl acrylate) or poly(dodecyl methacrylate)macromonomers comprising mono(meth)acrylate end groups; poly(stearyl acrylate) and poly(stearyl methacrylate)macromonomers comprising mono(meth)acrylate end groups.

Such macromonomers are, for example, described in European Patent Nos. EP 895 467 and EP 96459 and in the article Gillman, Polymer Letters, Vol. 5, page 477-481 (1967).

Non-limiting mention may also be made of macromonomers based on poly(2-ethylhexyl acrylate) or poly(dodecyl acrylate) comprising mono(meth)acrylate end groups.

(ii) polyolefins with an ethylenically unsaturated end group, for example, those with a (meth)acrylate end group. Examples of such polyolefins that may be mentioned, for example, comprise the following macromonomers, it being understood that they contain a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers, polybutadiene macromonomers; polyisoprene macromonomers; polybutadiene macromonomers; poly(ethylene/butylene)-polyisoprene macromonomers.

Such macromonomers are described, for example, in European Patent No. EP 1 347 013 or in U.S. Pat. No. 5,625,005, which mentions ethylene/butylene and ethylene/propylene macromonomers comprising a (meth)acrylate reactive end group. Non-limiting mention may also be made of poly(ethylene/butylene)methacrylate, such as the product sold under the name Kraton Liquid L-1253 by Kraton Polymers.

Among the soluble monomers that may be used, further non-limiting mention may be made of, for example:
the methacrylates of formula CH₂═C(CH₃)—COOR₁
wherein R¹ is chosen from linear and branched C8-C22 alkyl groups such as lauryl, behenyl or stearyl; cyclic alkyl groups comprising from 8 to 30 carbon atoms, such as isobornyl; and tert-butyl groups;
the acrylates of formula CH₂═CH—COOR₂
wherein R₂ is chosen from linear and branched C8-C22 alkyl groups such as lauryl, behenyl, 2-ethylhexyl or stearyl; cyclic alkyl groups comprising from 8 to 30 carbon atoms, such as isobomyl; and isobutyl groups; and mixtures thereof.

Non-limiting mention may thus be made of 2-ethylhexyl acrylate, isobornyl (meth)acrylate, lauryl(meth)acrylate, stearyl(meth)acrylate, behenyl(meth)acrylate, isobutyl acrylate and tert-butyl methacrylate, and mixtures thereof.

Among the insoluble monomers that may be used, non-limiting mention may be made, alone or as a mixture, of the following monomers, and also the salts and mixtures thereof;
(i) the (meth)acrylates chosen from formulae: CH₂═C(CH₃)—COOR′₁ and CH₂═CH—COOR′₁ wherein R′₁ is chosen from:

linear and branched alkyl groups comprising from 1 to 6 carbon atoms, said group optionally comprising in its chain at least one hetero atom chosen from O, N and S; optionally comprising at least one substituent chosen from —OH, halogen atoms (F, Cl, Br or I) and —NR′R″ groups wherein R′ and R″, which may be identical or different, are each chosen from linear and branched C1-C4 alkyls; optionally being substituted with at least one polyoxyalkylene group, for example, with a C2-C4 alkylene and, for example, polyoxyethylene and/or polyoxypropylene, said polyoxyalkylene group consisting of a repetition of from 5 to 30 oxyalkylene units; tert-butyl methacrylate and isobutyl acrylate are excluded from this definition;

cyclic alkyl groups comprising from 3 to 6 carbon atoms, said group optionally comprising in its chain at least one hetero atom chosen from O, N and S and/or optionally comprising at least one substituent chosen from OH and halogen atoms (F, Cl, Br or I).

Examples of R′1 that may be mentioned comprise, for example, methyl, ethyl, propyl, butyl, methoxyethyl, ethoxyethyl, methoxypolyoxyethylene 30, trifluoroethyl, 2-hydroxyethyl, 2-hydroxypropyl, dimethylaminoethyl, diethylaminoethyl and dimethylaminopropyl groups;
(ii) the (meth)acrylamides of formulae: CH₂═C(CH₃)—CONR′₃R′₄ and CH₂═CH—CONR′₃R′₄,
wherein:

R′3 and R′4, which may be identical or different, are each chosen from hydrogen atoms and linear and branched alkyl groups comprising from 1 to 6 carbon atoms, optionally comprising at least one substituent chosen from —OH, halogen atoms (F, Cl, Br or I) and —NR′R″ groups wherein R′ and R″, which may be identical or different, are each chosen from linear and branched C1-C4 alkyls; or, alternatively,

R′3 is chosen from a hydrogen atom and R′4 is chosen from a 1,1-dimethyl-3-oxobutyl group.

As examples of alkyl groups that can constitute R′3 and R′4, non-limiting mention may be made of n-butyl, t-butyl, n-propyl, dimethylaminoethyl, diethylaminoethyl and dimethylaminopropyl;

(iii) ethylenically unsaturated monomers comprising at least one carboxylic, phosphoric or sulfonic acid functional group, such as crotonic acid, maleic anhydride, itaconic acid, fumaric acid, maleic acid, styrenesulfonic acid, vinylbenzoic acid, vinylphosphoric acid, acrylic acid, methacrylic acid and acrylamidopropanesulfonic acid, and salts thereof;

(iv) the vinyl esters of formula: R′6-COO—CH═CH₂ wherein R′6 is chosen from linear and branched alkyl groups comprising from 1 to 6 atoms; cyclic alkyl groups comprising from 3 to 6 carbon atoms; and aromatic groups, for example of benzene, anthracene or naphthalene type;

(v) ethylenically unsaturated monomers comprising at least one tertiary amine functional group, such as 2-vinylpyridine or 4-vinylpyridine, and mixtures thereof;

(vi) styrene and derivatives thereof;

(vii) the di-n-alkylitaconates of formula:
CH₂═C(CH₂—COO(CH₂)ₙ₋₁—CH₃)—COO(CH₂)ₙ₋₁—CH₃, wherein n is chosen from an integer ranging from 0 to 4;

(viii) ethylenic monomers wherein the ester group contains silanes, silsesquioxanes, siloxanes or carbosiloxane dendrimers as described in European Patent No. EP 0 963 751, with the exception of monomers comprising only one silicon atom such as methacryloxypropyl trimethoxysilane. For example, the monomers can be chosen from: (meth)acryloxypropyltris(trimethylsiloxy)silane, (meth)acryloxypropylbis(trimethylsiloxy)methylsilane, (meth)acryloxymethyltris(trimethylsiloxy)silane and (meth)acryloxymethylbis(trimethylsiloxy)methylsilane;

(ix) PDMS macromonomers, such as polydimethylsiloxanes comprising monoacryloyloxy or monomethacryloyloxy end groups, and, for example, those comprising the following formula: 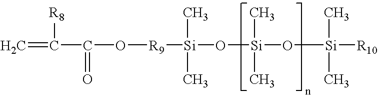
wherein:

R8 is chosen from a hydrogen atom and methyl groups; for example, methyl;

R9 is chosen from linear and branched, for example, linear, divalent hydrocarbon-based groups comprising from 1 to 10 carbon atoms and optionally comprising one or two ether bonds —O—; for example, ethylene, propylene or butylene;

R10 is chosen from linear and branched alkyl groups comprising from 1 to 10 carbon atoms and, for example, from 2 to 8 carbon atoms; for example, methyl, ethyl, propyl, butyl or pentyl;

n is chosen from an integer ranging from 1 to 300, for example, ranging from 3 to 200 and further, for example, ranging from 5 to 100.

Monomethacryloyloxypropyl polydimethylsiloxanes such as those sold under the name PS560-K6 by UCT (United Chemical Technologies Inc.) or under the name MCR-M17 by Gelest Inc. may be used, for example;

(x) oligopeptides functionalized with a (meth)acrylate functional group.

Among the salts that may be used as disclosed herein, non-limiting mention may be made of those obtained by neutralization of acidic groups using mineral bases such as sodium hydroxide, potassium hydroxide or ammonium hydroxide, or organic bases such as alkanolamines, for instance monoethanolamine, diethanolamine, triethanolamine or 2-methyl-2-amino-1-propanol.

Further non-limiting mention may also be made of the salts formed by neutralization of the tertiary amine units, for example using a mineral or organic acid. Among the mineral acids that may be mentioned include, for instance, sulfuric acid, hydrochloric acid, hydrobromic acid, hydroiodic acid, phosphoric acid and boric acid. Among the organic acids that may be mentioned include, for instance, acids comprising at least one carboxylic, sulfonic or phosphonic groups. These may be linear, branched or cyclic aliphatic acids or alternatively aromatic acids. These acids may also comprise at least one hetero atom chosen from O and N, for example, in the form of hydroxyl groups. Mention may be made, for example, of acetic acid, propionic acid and terephthalic acid, and also citric acid and tartaric acid.

Insoluble monomers that may be further mentioned comprise, for example:

the (meth)acrylates of formulae: CH₂═C(CH₃)—COOR′₁ and CH₂═CH—COOR′₁, for example, methyl, ethyl, propyl or butyl(meth)acrylate; isobutyl methacrylate; methoxy ethyl or ethoxy ethyl(meth)acrylate; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate;

ethylenically unsaturated monomers comprising at least one carboxylic acid functional group, for example, (meth)acrylic acid and salts thereof;

maleic anhydride;

ethylenic monomers wherein the ester group contains silanes;

polydimethylsiloxanes comprising a monoacryloyloxy or monomethacryloyloxy end group, comprising the following formula: 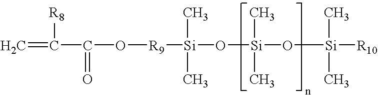
wherein:

R8 is chosen from a hydrogen atom and methyl groups; for example, methyl;

R9 is chosen from linear and branched, for example, linear, divalent hydrocarbon-based groups comprising from 1 to 10 carbon atoms and optionally comprising one or two ether bonds —O—; for example, ethylene, propylene or butylene;

R10 is chosen from linear and branched alkyl groups comprising from 1 to 10 carbon atoms and, for example, from 2 to 8 carbon atoms;, for example, methyl, ethyl, propyl, butyl or pentyl;

n is chosen from integers ranging from 1 to 300, for example, ranging from 3 to 200 and further, for example, ranging from 5 to 100.

Non-limiting mention may be made, for further example, of methyl (meth)acrylate, ethyl(meth)acrylate, (meth)acrylic acid, maleic anhydride, (meth)acryloxypropyltris(trimethylsiloxy)silane, (meth)acryloxypropylbis(trimethylsiloxy)methylsilane, (meth)acryloxymethyltris(trimethylsiloxy)silane and (meth)acryloxymethylbis(trimethylsiloxy)methylsilane.

The at least one polymer particle dispersion according to the disclosure also comprises a liquid carbon-based medium wherein said particles are dispersed.

As used herein, the term “liquid medium”, for example, is understood to mean a medium, for instance, having a viscosity of less than or equal to 7000 centipoises at 20° C.

According to the disclosure, the medium is said to be carbon-based if it comprises at least 50% by weight, for example, from 50% to 100% by weight, such as from 60% to 99% by weight or further, for example, from 65% to 95% by weight, or even from 70% to 90% by weight, relative to the total weight of the carbon-based medium, of carbon-based compound that is liquid at 25° C., having a global solubility parameter according to the Hansen solubility space of less than or equal to 20 (MPa)^{1/2}, or a mixture of such compounds.

The global solubility parameter δ according to the Hansen solubility space is defined in the article “Solubility parameter values” by Grulke, in the book “Polymer Handbook” 3rd Edition, Chapter VII, pages 519-559, by the relationship:
δ=(d_{D}²+d_{P}²+d_{H}²)^{1/2}
wherein:

d_{D} is the London dispersion forces derived from the formation of dipoles induced during molecular impacts,

d_{P} is the Debye interaction forces from permanent dipoles,

d_{H} is the specific interaction forces (such as hydrogen bonding, acid/base bonding, donor/acceptor bonding, etc.).

The definition of solvents in the three-dimensional solubility space according to Hansen is described in Hansen's article: “The three dimensional solubility parameters” J. Paint Technol. 39, 105 (1967).

Among the liquid carbon-based mediums with a global solubility parameter according to the Hansen solubility space of less than or equal to 20 (MPa)^{1/2}, non-limiting mention may be made of liquid fatty substances, for example, oils, which may be chosen from natural or synthetic, carbon-based or hydrocarbon-based oils, which are optionally fluorinated, and which are optionally branched, alone or as a mixture.

Non-limiting mention may be made, for example, of:

plant oils formed by fatty acid esters of polyols, for example, triglycerides, such as sunflower oil, sesame seed oil, rapeseed oil, macadamia oil, soybean oil, sweet almond oil, beauty-leaf oil, palm oil, grape seed oil, corn oil, arara oil, cottonseed oil, apricot oil, avocado oil, jojoba oil, olive oil or cereal germ oil;

linear, branched or cyclic esters, comprising more than 6 carbon atoms, for example, 6 to 30 carbon atoms; for example, isononyl isononanoate; and further, for example, the esters of formula RCOOR′ wherein R is chosen from a higher fatty acid residue comprising from 7 to 19 carbon atoms and R′ is chosen from a hydrocarbon-based chain comprising from 3 to 20 carbon atoms, such as palmitates, adipates, myristates and benzoates, for example, diisopropyl adipate and isopropyl myristate;

hydrocarbons, for example, volatile or non-volatile, linear, branched and/or cyclic alkanes, such as optionally volatile C₅-C₆₀ isoparaffins such as isododecane, Parleam (hydrogenated polyisobutene), isohexadecane, cyclohexane, or “Isopar” products; or alternatively liquid paraffin, liquid petroleum jelly or hydrogenated polyisobutylene;

ethers comprising more than 6 carbon atoms, for example, 6 to 30 carbon atoms;

ketones comprising more than 6 carbon atoms, for example, 6 to 30 carbon atoms;

aliphatic fatty monoalcohols comprising 6 to 30 carbon atoms, the hydrocarbon-based chain not comprising any substitution groups, such as oleyl alcohol, decanol, dodecanol, octadecanol, octyldodecanol and linoleyl alcohol;

polyols, for example, comprising 6 to 30 carbon atoms, such as hexylene glycol; and

mixtures thereof.

For example, the dispersion comprises in the carbon-based medium at least one carbon-based compound chosen from:

plant oils formed from fatty acid esters of polyols, for example, triglycerides;

the esters of formula RCOOR′ wherein R is chosen from a higher fatty acid residue comprising from 7 to 19 carbon atoms and R′ is chosen from a hydrocarbon-based chain comprising from 3 to 20 carbon atoms;

volatile and non-volatile linear and branched C₈-C₆₀ alkanes;

volatile and non-volatile non-aromatic cyclic C5-C12 alkanes;

ethers comprising 7 to 30 carbon atoms;

ketones comprising 8 to 30 carbon atoms; and

aliphatic fatty monoalcohols comprising 12 to 30 carbon atoms, the hydrocarbon-based chain not comprising any substitution groups.

A dispersion comprising polymer particles in such a limited carbon-based medium is novel and as such constitutes an embodiment of the present disclosure.

The carbon-based medium may, for example, comprise as carbon-based compounds: isopropyl myristate, octyldodecanol, C5-C60 isoparaffins, isohexadecane or isononyl isononanoate.

The carbon-based medium may optionally comprise additional liquid compounds that may be present in an amount of strictly less than 50% by weight, for example, from 1% to 40% by weight, further, for example, from 5% to 35% by weight, even further, for example, from 10% to 30% by weight, relative to the total weight of the carbon-based medium, and chosen, alone or as a mixture, from:

volatile and non-volatile silicone oils, alone or as a mixture.

Non-limiting mention may be made, for example, of polydimethylsiloxanes and polymethylphenylsiloxanes, optionally substituted with aliphatic and/or aromatic groups, which are optionally fluorinated, and/or comprising functional groups such as hydroxyl, thiol and/or amine groups; and volatile silicones, for example, cyclic and linear volatile silicones, such as cyclodimethylsiloxanes, cyclophenylmethylsiloxanes and linear dimethylsiloxanes, among which non-limiting mention may be made of linear dodecamethylpentasiloxane (L5), octamethylcyclotetrasiloxane, decamethylcyclopentasiloxane, hexadecamethylcyclohexasiloxane, heptamethylhexyltrisiloxane and heptamethyloctyltrisiloxane,

esters comprising from 2 to 5 carbon atoms, ethers comprising from 2 to 6 carbon atoms, ketones comprising from 1 to 5 carbon atoms, and monoalcohols comprising from 1 to 5 carbon atoms.

However, according to one embodiment of the disclosure, the carbon-based medium does not contain, for example, any additional liquid compounds.

The choice of the carbon-based medium may be readily made by a person skilled in the art as a function of the nature of the monomers constituting the polymer and/or of the intended use of the composition.

For example, mention may be made of dispersions of poly(2-ethylhexyl acrylate)-b-p(methyl acrylate), poly(isobornyl acrylate)-b-poly(methyl acrylate), or poly(2-ethylhexyl acrylate-co-isobornyl acrylate)-b-poly(methyl acrylate) particles, in, for instance an alkane and, for example, in isododecane.

Non-limiting mention may also be made of dispersions of the following polymers, for example, in alkanes and, further, for example, in isododecane:

poly(2-ethylhexyl acrylate)-b-poly(methyl acrylate-co-acrylic acid),

poly(2-ethylhexyl acrylate-co-acrylic acid)-b-poly(methyl acrylate),

poly(2-ethylhexyl acrylate-co-isobornyl acrylate-co-acrylic acid)-b-poly(methyl acrylate),

poly(isobornyl acrylate)-b-poly(methyl acrylate-co-acrylic acid),

poly(isobornyl acrylate)-b-poly(methyl acrylate-co-acrylic acid),

poly(isobornyl acrylate-co-acrylic acid)-b-poly(methyl acrylate),

poly(isobutyl acrylate)-b-poly(methyl acrylate),

poly(isobutyl acrylate)-b-poly(methyl acrylate-co-acrylic acid),

poly(isobutyl acrylate-co-acrylic acid)-b-poly(methyl acrylate),

poly(isobutyl acrylate-co-isobornyl acrylate)-b-poly(methyl acrylate),

poly(isobutyl acrylate-co-isobornyl acrylate)-b-poly(methyl acrylate-co-acrylic acid),

poly(2-ethylhexyl acrylate)-b-poly(methyl acrylate)-b-poly(2-ethylhexyl acrylate),

poly(2-ethylhexyl acrylate-co-isobornyl acrylate)-b-poly(methyl acrylate)-b-poly(2-ethylhexyl acrylate-co-isobornyl acrylate),

poly(2-ethylhexyl acrylate-co-acrylic acid)-b-poly(methyl acrylate)-b-poly(2-ethylhexyl acrylate-co-acrylic acid),

poly(2-ethylhexyl acrylate)-b-poly(methyl acrylate-co-acrylic acid)-b-poly(2-ethylhexyl acrylate),

poly(2-ethylhexyl acrylate-co-isobornyl acrylate-co-acrylic acid)-b-poly(methyl acrylate)-b-poly(2-ethylhexyl acrylate-co-isobornyl acrylate-co-acrylic acid), and

poly(2-ethylhexyl acrylate-co-isobomyl acrylate)-b-poly(methyl acrylate-co-acrylic acid)-b-poly(2-ethylhexyl acrylate-co-isobornyl acrylate).

The dispersion according to the disclosure, for example, can have a solids content ranging from 5% and 80% by weight, for example, from 8% to 70% by weight, further, for example, from 10% to 60%, or even further, for example, from 15% to 50% by weight and from 18% to 25% by weight.

The polymer dispersion may be manufactured by any means known to those skilled in the art, and, for example, by controlled free-radical polymerization or by living polymerization, for example, via the nitroxide/alkoxyamine, ATRP, organocobalt, RAFT/MADIX, degenerative transfer, TERP (tellurium) or selenium techniques, via Iniferter, or via any living polymerization process (anionic or cationic), via metallocene, ROMP (ring-opening metathesis polymerization), cationic or anionic ROP (ring-opening polymerization), GTP (group-transfer polymerization), tetraphenylethane derivatives or diphenylethylene. The techniques used for the formation of each block may be identical or different.

A typical process may consist in preparing the first block, referred to as the soluble block, in the carbon-based dispersion medium, by polymerization of the monomer(s), a control agent and an initiator, if necessary. Next, the monomer(s) of the “insoluble” block is(are) added in the presence or absence of initiator. The reaction temperature is, for example, from −30 to 200° C., for example, from 0 to 160° C. and further, for example, from 40 to 140° C. Additional blocks may be polymerized according to the same process. For each of the blocks, the monomer(s) may be added simultaneously, in batch mode, semi-continuously or consecutively. Multiblock polymers will then be obtained.

If the first block, referred to as the soluble block, is synthesized in bulk, the “insoluble” block may then be synthesized in bulk or in solution. The solvent may be a carbon-based solvent as defined in the present patent application, which leads at the end of the synthesis of the copolymer to a dispersion directly in the carbon-based medium. The solvent used may also be a solvent common to all the blocks; in this case, the subsequent addition of a carbon-based solvent as defined above in the present patent application the optional removal of the common solvent will lead to the disclosed dispersion in the carbon-based medium.

If the whole copolymer is synthesized in bulk, the addition of a carbon-based solvent as defined above will lead to the dispersion embodied herein.

If all the blocks are synthesized in solution, in a common solvent, the subsequent addition of a carbon-based solvent as defined above and the optional removal of the common solvent will lead to the disclosed dispersion in the carbon-based media. It is also possible at this stage to remove the common solvent in order to recover the polymer alone and before dispersing it in a carbon-based solvent as defined above, which will lead to the disclosed dispersion.

Finally, if all the blocks are synthesized directly in a carbon-based solvent as defined above, the dispersion is obtained directly, in a single step.

Once the dispersion has been obtained, it is possible to change the carbon-based medium by removal/addition of a new carbon-based solvent or by addition/optional removal of the first solvent.

For example, if the first block is prepared by controlled radical polymerization (CRP), then, in one embodiment, the second block can also optionally be prepared by CRP or by conventional polymerization.

Another embodiment disclosed herein consists in synthesizing the soluble block in bulk, and then in dissolving it in a carbon-based solvent according to the disclosure, and then in synthesizing the insoluble block in this carbon-based solvent; a dispersion of the polymer in the carbon-based solvent is thus directly obtained.

In yet another embodiment of the disclosure, once the dispersion has been obtained, it is possible to add thereto at least one monomer C whose homopolymers are either of soluble type or of insoluble type, according to the definition given above, and, for example, in one embodiment, insoluble, in the medium, in order to continue the polymerization on the copolymers comprising blocks A-B already formed, which leads to the formation of triblock copolymers A-B-C.

The additional monomer(s) C may be present in an amount such that the total amounts of soluble and insoluble monomers remain within the total ranges mentioned above.

When the starting copolymer is a triblock copolymer of structure A-B-A, the polymerization of C may lead to a pentablock copolymer of structure C-A-B-A-C or A-B-C-B-A, depending on the polymerization technique and/or the transfer agent used.

The polymerization initiator may be an initiator known to those skilled in the art for free-radical polymerization (peroxides, azo compounds, redox couple or photochemical initiator). In the case of certain controlled radical polymerization techniques, the same compound may have the role of polymerization initiator and may be a control agent, as is the case for alkoxyamines. For non-radical polymerizations, i.e. ionic (anionic or cationic) polymerizations, a person skilled in the art can select the appropriate initiator.

Copolymers that self-organize in dispersion in the medium under consideration are thus obtained. They are composed of a first soluble block A and of at least one second, insoluble block, B, which will cause self-organization of the polymer chains so as to form particles having at the interface with the medium the blocks A and at the core of the particle the blocks B. Once the dispersion has been obtained, it is possible to add dispersants or stabilizers thereto in order to modify its physicochemical properties (viscosity, Tg, etc.).

The dispersions according to the disclosure may find application in cosmetics. Thus, they may be present in the cosmetic compositions according to the disclosure in an amount ranging from 0.1% to 90% by weight, for example, from 0.5% to 80% by weight, further, for example, from 1% to 75% by weight and, from 5% to 70% by weight of dispersion, relative to the total weight of the composition.

The cosmetic compositions according to the disclosure also comprise a cosmetically acceptable medium, i.e. a medium that is compatible with keratin materials such as facial or body skin, the lips, the hair, the eyelashes, the eyebrows and the nails.

The composition may, for example, comprise at least one fatty phase, which may itself comprise at least one oil and/or solvent, which are, for example, lipophilic, and also fatty substances that are solid at room temperature, such as waxes, pasty fatty substances and gums, and mixtures thereof.

Among the constituents of the at least one fatty phase that may be mentioned, by way of non-limiting example, are volatile or non-volatile oils, which may be chosen from carbon-based, hydrocarbon-based, fluorinated, optionally branched, natural or synthetic oils, alone or as a mixture. As used herein, the term “non-volatile oil” is understood to mean an oil that is capable of remaining on the skin at room temperature and atmospheric pressure for at least one hour and, for example, having a non-zero vapor pressure at room temperature (25° C.) and atmospheric pressure, of less than 0.01 mm Hg (1.33 Pa).

Non-limiting mention may be made of non-volatile carbon-based and, for example, hydrocarbon-based oils, of plant, mineral, animal or synthetic origin, such as liquid paraffin (or petroleum jelly), squalane, hydrogenated polyisobutene (Parleam), perhydrosqualene, mink oil, macadamia oil, turtle oil, soybean oil, sweet almond oil, beauty-leaf oil, palm oil, grape seed oil, sesame seed oil, corn oil, arara oil, rapeseed oil, sunflower oil, cottonseed oil, apricot oil, castor oil, avocado oil, jojoba oil, olive oil or cereal germ oil, and shea butter; linear, branched or cyclic esters comprising more than 6 carbon atoms and, for example, 6 to 30 carbon atoms, such as lanolic acid, oleic acid, lauric acid or stearic acid esters; esters derived from long-chain acids or alcohols (i.e. comprising from 6 to 20 carbon atoms), for example, the esters of formula RCOOR′ wherein R is chosen from a higher fatty acid residue comprising from 7 to 19 carbon atoms and R′ is chosen from a hydrocarbon-based chain comprising from 3 to 20 carbon atoms, for example, C12-C36 esters such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyidodecyl myristate or lactate, bis(2-ethylhexyl)succinate, diisostearyl malate, and glyceryl or diglyceryl triisostearate; higher fatty acids, for example, of C14-C22, such as myristic acid, palmitic acid, stearic acid, behenic acid, oleic acid, linoleic acid, linolenic acid or isostearic acid; higher fatty alcohols, for example, of C16-C22, such as cetanol, oleyl alcohol, linoleyl alcohol, linolenyl alcohol, isostearyl alcohol or octyldodecanol; and mixtures thereof.

Non-limiting mention may also be made of decanol, dodecanol, octadecanol, liquid triglycerides of fatty acids of 4 to 10 carbon atoms, for instance heptanoic or octanoic acid triglycerides, caprylic/capric acid triglycerides; linear or branched hydrocarbons, of mineral or synthetic origin, such as liquid paraffins and derivatives thereof, petroleum jelly, polydecenes and hydrogenated polyisobutene, such as Parleam; synthetic esters and ethers, for example, of fatty acids, for instance, Purcellin oil, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols comprising from 12 to 26 carbon atoms, for instance octyldodecanol, 2-butyloctanol, 2-hexyldecanol or 2-undecylpentadecanol.

Non-limiting mention may also be made of ketones that are liquid at room temperature, such as methyl ethyl ketone, methyl isobutyl ketone, diisobutyl ketone, isophorone, cyclohexanone or acetone; propylene glycol ethers that are liquid at room temperature, such as propylene glycol monomethyl ether, propylene glycol monomethyl ether acetate or dipropylene glycol mono-n-butyl ether; short-chain esters (comprising from 3 to 8 carbon atoms in total), such as ethyl acetate, methyl acetate, propyl acetate, n-butyl acetate or isopentyl acetate; ethers that are liquid at room temperature, such as diethyl ether, dimethyl ether or dichlorodiethyl ether; alkanes that are liquid at room temperature, such as decane, heptane, dodecane, isododecane, isohexadecane or cyclohexane; aromatic cyclic compounds that are liquid at room temperature, such as toluene and xylene; aldehydes that are liquid at room temperature, such as benzaldehyde and acetaldehyde, and mixtures thereof.

Among the volatile compounds that may be used, non-limiting mention may be made of non-silicone volatile oils, for example, C8-C16 isoparaffins, for instance isododecane, isodecane and isohexadecane. Non-limiting mention may be made of volatile or non-volatile alkanes that are liquid at room temperature, for example, decane, heptane, dodecane, isododecane, isohexadecane, cyclohexane and isodecane, and mixtures thereof.

The fatty phase may be present in an amount ranging from 0.01% to 95%, for example, from 0.1% to 90%, further, for example, from 10% to 85% and even further, for example, from 30% to 80% by weight, relative to the total weight of the composition.

The composition may also comprise at least one hydrophilic phase comprising water or a mixture of water and at least one hydrophilic organic solvent, for instance alcohols and, for example, linear and branched lower monoalcohols comprising from 2 to 5 carbon atoms, for instance ethanol, isopropanol or n-propanol, and polyols, for instance glycerol, diglycerol, propylene glycol, sorbitol, pentylene glycol, and polyethylene glycols, or alternatively hydrophilic C₂ ethers and hydrophilic C₂-C₄ aldehydes. Water or the mixture of water and at least one hydrophilic organic solvent may be present in the composition according to the disclosure in an amount ranging from 0.1% to 80% by weight, for example, from 1% to 70% by weight, relative to the total weight of the composition.

The composition according to the disclosure may also comprise at least one wax and/or gum. As used herein, the term “wax” is understood to mean a lipophilic compound that is solid at room temperature (25° C.), with a reversible solid/liquid change of state, having a melting point of greater than or equal to 30° C., which may be up to 120° C. By bringing the wax to the liquid state (melting), it is possible to make it miscible with the oils that may be present and to form a microscopically homogeneous mixture, but on returning the temperature of the mixture to room temperature, recrystallization of the wax in the oils of the mixture is obtained. The melting point of the wax may be measured using a differential scanning calorimeter (DSC), for example the calorimeter sold under the name DSC 30 by the company Mettler.

The at least one wax may be chosen from hydrocarbon-based waxes, fluoro waxes and/or silicone waxes and may be of plant, mineral, animal and/or synthetic origin. For example, the at least one wax can have a melting point of greater than 25° C. and further, for example, greater than 45° C. Among the waxes that may be used in the composition of the disclosure, non-limiting mention may be made of beeswax, carnauba wax or candililla wax, paraffin, microcrystalline waxes, ceresin or ozokerite; synthetic waxes, for instance polyethylene waxes or Fischer Tropsch waxes, and silicone waxes, for instance alkyl or alkoxy dimethicones comprising from 16 to 45 carbon atoms.

The at least one gum can be chosen from high molecular weight polydimethylsiloxanes (PDMSs) and cellulose and polysaccharide gums, and the at least one pasty substance can be chosen from hydrocarbon-based compounds, for instance lanolins and derivatives thereof, or alternatively PDMSs.

The nature and amount of the solid substances depend on the desired mechanical properties and textures. As a guide, the composition may contain from 0.01% to 50% by weight and further, for example, from 1% to 30% by weight of waxes, relative to the total weight of the composition.

The composition according to the disclosure may also comprise at least one dyestuff chosen from water-soluble dyes, liposoluble dyes and pulverulent dyestuffs, for instance pigments, nacres and flakes that are well known to those skilled in the art. The at least one dyestuff may be present in the composition in an amount ranging from 0.01% to 50% by weight, for example, from 0.01% to 30% by weight, relative to the weight of the composition.

As used herein, the term “pigments” is understood to mean white or colored, mineral or organic particles of any form, which are insoluble in physiological medium and are intended to color the composition. As used herein, the term “nacres” is understood to mean iridescent particles of any form, for example, produced by certain mollusks in their shell or else synthesized. The pigments may be white or colored, and mineral and/or organic. Among the mineral pigments that may be mentioned, in a non-limiting manner, are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder. Among the organic pigments that may be mentioned, in a non-limiting manner, are carbon black, pigments of D & C type, and lakes based on cochineal carmine or on barium, strontium, calcium or aluminium. The nacreous pigments may be chosen from white nacreous pigments such as mica coated with titanium or with bismuth oxychloride, colored nacreous pigments such as titanium mica coated with iron oxides, titanium mica coated, for example, with ferric blue or with chromium oxide, titanium mica coated with an organic pigment of the abovementioned type, and also nacreous pigments based on bismuth oxychloride.

Among the water-soluble dyes that may be mentioned, non-limiting examples include the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll and methylene blue.

The composition according to the disclosure may also comprise at least one filler, for example, present in an amount ranging from 0.01% to 50% by weight, for example, ranging from 0.01% to 30% by weight, relative to the total weight of the composition. As used herein, the term “fillers” is understood to mean colorless or white, mineral or synthetic particles of any form, which are insoluble in the medium of the composition irrespective of the temperature at which the composition is manufactured. These fillers serve, for example, to modify the rheology or texture of the composition. The fillers may be mineral or organic of any form, platelet-shaped, spherical or oblong, irrespective of the crystallographic form (for example lamellar, cubic, hexagonal, orthorhombic, etc.). Non-limiting mention may be made of talc, mica, silica, kaolin, polyamide (Nylon®) powders (Orgasol® from Atochem), poly-β-alanine powders and polyethylene powders, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids comprising from 8 to 22 carbon atoms, for example, from 12 to 18 carbon atoms, for example zinc stearate, magnesium stearate, lithium stearate, zinc laurate or magnesium myristate.

The composition may also comprise at least one additional polymer such as a film-forming polymer. According to the present disclosure, the term “film-forming polymer” is understood to mean a polymer that is capable, by itself or in the presence of an auxiliary film-forming agent, of forming a continuous film that adheres to a support and, for example, to keratin materials. Among the film-forming polymers that may be used in the composition of the present disclosure, non-limiting mention may be made of synthetic polymers, of free-radical type or of polycondensate type, polymers of natural origin, and mixtures thereof, for example, acrylic polymers, polyurethanes, polyesters, polyamides, polyureas and cellulose-based polymers, for instance nitrocellulose.

The composition according to the disclosure may also comprise at least one adjuvant commonly used in cosmetics, such as vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides, or mixtures thereof. A person skilled in the art will take care to select this or these optional additional compound(s), and/or the amount thereof, such that the beneficial properties of the composition according to the disclosure are not, or are not substantially, adversely affected by the envisaged addition.

The composition according to the disclosure may, for example, be in the form of a suspension, a dispersion, a solution, for example, an organic solution, a gel, an emulsion, for example, an oil-in-water (O/W) or water-in-oil (W/O) emulsion, or a multiple emulsion (W/O/W, polyol/O/W or O/W/O emulsion), or in the form of a cream, a paste, a mousse, a dispersion of vesicles, for example, of ionic or nonionic lipids, a two-phase or multi-phase lotion, a spray, a powder or a paste, for example, a soft paste (for example, a paste with a dynamic viscosity at 25° C. of about from 0.1 to 40 Pa.s at a shear rate of 200 s⁻¹, after 10 minutes of measurement in cone/plate geometry). The composition may be anhydrous, for example it may be an anhydrous paste.

A person skilled in the art may select the appropriate galenical form, and also the method for preparing it, on the basis of his general knowledge, taking into account firstly the nature of the constituents used, for example, their solubility in the support, and secondly the intended use of the composition.

The composition according to the disclosure may be a makeup composition, for example, a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

The composition according to the disclosure may be a composition for protecting or caring for the skin of the face, the neck, the hands or the body, for example, an anti-wrinkle or anti-fatigue composition for making the skin look radiant, or a moisturizing or treating composition; an anti-sun or self-tanning composition.

The composition according to the disclosure may also be a hair product, for example, for holding the hairstyle or for shaping the hair. The hair compositions are, for example, shampoos, hair setting gels or lotions, blow-waving lotions, or fixing and styling compositions such as lacquers or sprays. The lotions may be packaged in various forms, for example, in vaporizers or pump-dispenser bottles or in aerosol containers in order to apply the composition in vaporized form or in the form of a mousse. Such packaging forms are indicated, for example, when it is desired to obtain a spray or a mousse for fixing or treating the hair.

The present disclosure also relates to a cosmetic process for making up, cleansing, protecting against the sun, shaping, dyeing or caring for keratin materials, for example, body or facial skin, the nails, the hair and/or the eyelashes, comprising the application to said materials of a cosmetic composition as defined above.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contains certain errors necessarily resulting from the standard deviation found in their respective testing measurements.

The embodiments disclosed herein are illustrated in greater detail by the non-limiting examples described below.

### EXAMPLES

### Examples 1 to 4

1/Synthesis of the Soluble Block

The various components of the mixture (monomer, transfer agent and initiator) were placed in contact and homogenized by stirring in a Rotaflo® round-bottomed flask equipped with a septum. The flask was then introduced into liquid nitrogen and placed under vacuum for one minute once the mixture had frozen. The mixture was then thawed and released the residual oxygen. This freezing/thawing cycle was repeated five times so as to remove the oxygen from the mixture. The flask was then introduced into an oil bath thermostatically maintained at 80° C. It was removed therefrom and cooled under a stream of cold water after variable reaction times. The blocks thus obtained, i.e. the functionalized poly(2-ethylhexyl acrylate)s, either TBD (tert-butyl dithiobenzoate) or TTC (bis(2-ethylhexyl)-2,2′-(trithiocarbonate)dipropanoate), were precipitated twice from cold methanol and then dried under a bell jar at reduced pressure.

| | |
|---|---|
| | structure of TBD |
| 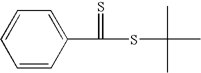 | |
| | structure of TTC |
| 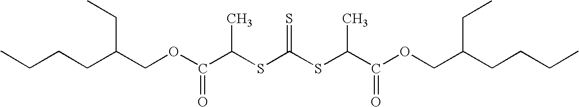 | |

| Block | | Transfer | | | Degree of | Theoretical | Exp. Mn |
|---|---|---|---|---|---|---|---|
| 1a | Monomer | agent | Initiator | Time | conversion | Mn* | (g/mol)/Ip |
| nature | 2-ethyl- | TBD | T21S | | | | 18 200/1.14 |
| | hexyl | | | | | | |
| | acrylate | | | | | | |
| mass | 61.52 g | 647.3 mg | 222.5 mg | 4 h | 82.5% | 16 500 | |
| Block | | Transfer | | | Degree of | Theoretical | Exp. Mn |
| 1b | Monomer | agent | Initiator | Time | conversion | Mn* | (g/mol)/Ip |
| nature | 2-ethyl- | TBD | T21S | | | | 12 600/1.14 |
| | hexyl | | | | | | |
| | acrylate | | | | | | |
| mass | 15.39 g | 164.6 mg | 57.3 mg | 2 h | 58% | 11 500 | |
| Block | | Transfer | | | Degree of | Theoretical | Exp. Mn |
| 1c | Monomer | agent | Initiator | Time | conversion | Mn* | (g/mol)/Ip |
| nature | 2-ethyl- | TTC | T21S | | | | 21 000/1.15 |
| | hexyl | | | | | | |
| | acrylate | | | | | | |
| mass | 15.38 g | 368.9 mg | 17.1 mg | 4 h | 80.2% | 16 000 | |
| Block | | Transfer | | | Degree of | Theoretical | Exp. Mn |
| 1d | Monomer | agent | Initiator | Time | conversion | Mn* | (g/mol)/Ip |
| nature | 2-ethyl- | TTC | T21S | | | | 15 000/1.65 |
| | hexyl | | | | | | |
| | acrylate | | | | | | |
| mass | 30.77 g | 737.8 mg | 33.1 mg | 4 h | 83% | 16 500 | |

*Theoretical Mn in g/mol, to the experimentally obtained conversion of the soluble block
**T21S: Trigonox 21S, tert-butyl peroxy-2-ethylhexanoate

2/Synthesis of the Insoluble Block, Formation of Dispersions

The various components of the mixture (monomer, soluble block, initiator and solvent) were placed in contact and homogenized by stirring in a Rotaflo® round-bottomed flask equipped with a septum. The flask was then introduced into liquid nitrogen and placed under vacuum for one minute once the mixture had frozen. The mixture was then thawed and released the residual oxygen. This freezing/thawing cycle was repeated five times so as to remove the oxygen from the mixture. The flask was then introduced into an oil bath thermostatically maintained at 80° C. It was removed therefrom and cooled under a stream of cold water after variable reaction times. A dispersion of block copolymers was thus obtained.

Examples 1 and 2 concern diblock copolymers. Examples 3 and 4 concern triblock copolymers of soluble-insoluble-soluble type.

| | Soluble | | | | Time/ | Theoretical | Exp. Mn*** |
|---|---|---|---|---|---|---|---|
| | block | Initiator | Monomer | Solvent | conversion | Mn** | (g/mol)/Ip |
| Ex. 1 | | | | | | | |
| nature | Block | T21S | Methyl | Isodo- | | | 28 000/6 |
| | 1a | | acrylate | decane | | | |
| | | | | 10 g | | | |
| mass | 1.5 g | 105.6 mg* | 3.05 g | | 24 h/70% | 27 000 | |
| Ex. 2 | | | | | | | |
| nature | Block | T21S | Methyl | Isodo- | | | 168 000/4.25 |
| | 1b | | acrylate | decane | | | |
| Mass | 1.5 g | 106.2 mg* | 4.47 g | 15 g | 24 h/69% | 26 400 | |
| Ex. 3 | | | | | | | |
| nature | Block | T21S | Methyl | Isodo- | | | 28 500/1.34 |
| | 1c | | acrylate | decane | | | |
| mass | 1.5 g | 109.3 mg* | 2.62 g | 8.6 g | 2 h/70% | 25 900 | |
| Ex. 4 | | | | | | | |
| nature | Block | T21S | Methyl | Isodo- | | | 25 100/3.33 |
| | 1d | | acrylate | decane | | | |
| mass | 1 g | 102.9 mg* | 2.42 g | 8.2 g | 4 h/62% | 23 600 | |

*Ex. 1: solution of T21S at 2.6 × 10⁻¹ mol/L in methyl acrylate
*Ex. 2: solution of T21S at 3.8 × 10⁻¹ mol/L in methyl acrylate
*Ex. 3: solution of T21S at 2.3 × 10⁻¹ mol/L in methyl acrylate
*Ex. 4: solution of T21S at 2.1 × 10⁻¹ mol/L in methyl acrylate
**Theoretical Mn of the insoluble block in g/mol, at the experimentally obtained conversion
***Experimental Mn of the insoluble block in g/mol, Ip of the block copolymer

### Example 5

2-Ethylhexyl acrylate (4 g), TBD (tert-butyl dithiobenzoate, 52.5 mg) and Trigonox 21S (18 mg) were placed in contact and homogenized by stirring in a Rotaflo® round-bottomed flask equipped with a septum. The flask was then introduced into liquid nitrogen and placed under vacuum for one minute once the mixture had frozen. The mixture was then thawed and released the residual oxygen. This freezing/thawing cycle was repeated five times so as to remove the oxygen from the mixture. The flask was then introduced into an oil bath thermostatically maintained at 80° C. After reaction for six hours, a sample was taken by syringe (block 1e) and a methyl acrylate (6.72 g)/T21S (17.5 mg)/isododecane (21.45 g) mixture, degassed beforehand by sparging with nitrogen (30 minutes), was then introduced by cannula. The flask was left for a further 22 hours in the bath at 80° C. The flask was then cooled under a stream of cold water. A dispersion of polymer particles in isododecane was obtained.

| | Theoretical Mn | Exp Mn*** (g/mol) | Ip |
|---|---|---|---|
| Block 1e | 17 000* | 17 000 | 1.13 |
| Dispersion | 25 300** | 55 600 | 6 |

*Theoretical Mn in g/mol of the soluble block, at total conversion
**Theoretical Mn in g/mol of the insoluble block, at total conversion
***Experimental Mn in g/mol for each of the blocks

### Example 6

### Characterization of the Dispersions of Examples 1 to 5

| | | Particle | | |
|---|---|---|---|---|
| | Dry | diameter | Mass % of | Mol % of |
| Example | extract | (PDI) | soluble fraction | soluble fraction |
| 1 | 28% | 50 nm | 39.4% | 23.2% |
| | | (0.22) | | |
| 2 | 24% | 80 nm | 7% | 3.4% |
| | | (0.10) | | |
| 3 | 30% | 35 nm | 42.4% | 25.7% |
| | | (0.09) | | |
| 4 | 24% | 50 nm | 37.4% | 21.9% |
| | | (0.07) | | |
| 5 | 28% | 40 nm | 23.1% | 12.2% |

PDI: Polydispersity index, which reflects the size polydispersity of the particles

The conversions were measured by ¹H NMR, for the soluble blocks, and by gravimetry for the insoluble blocks.

The molar masses were determined by GPC in THF with linear polystyrene standards.

The mean particle diameters were measured by dynamic light scattering with a Malvern Nano-S90 machine, taking into account the refractive index and the viscosity of the solvent.

### Example 7

### Mascara Composition

A mascara comprising the composition below was prepared:

| | | |
|---|---|---|
| Waxes | 17 | g |
| Modified hectorite (Bentone ® 38V from Elementis) | 5.3 | g |
| Propylene carbonate | 1.7 | g |
| Filler | 1 | g |
| Pigments | 5 | g |
| Polymer dispersion of Example 1 | 10 | g DM* |
| Isododecane | qs 100 | g |

*DM: dry matter

The mascara, after application to the eyelashes, was judged very satisfactory.

### Example 8

### Stick of Lipstick

The lipstick composition below was prepared:

| | | |
|---|---|---|
| Wax | 15% | |
| Dispersion of polymer of Example 2 | 10% | DM |
| Non-volatile carbon-based oil | 26% | |
| Pigments | 8.6% | |
| Isododecane | qs 100% | |

The composition obtained after application to the lips had good cosmetic properties.

### Example 9

### W/O Foundation

A foundation composition comprising the compounds that follow was prepared:

| Phase A | | |
|---|---|---|
| Cetyl dimethicone copolyol | 3 | g |
| (Abil EM 90 from the company Goldschmidt) | | |
| Isostearyl diglyceryl succinate | 0.6 | g |
| (Imwitor 780K from the company Condea) | | |
| Isododecane | 18.5 | g |
| Pigments | 10 | g |
| (hydrophobic iron oxides and titanium oxides) | | |
| Polymer dispersion of Example 3 | 8 | g DM |
| Filler | 8 | g |
| Fragrance | qs | |

| Phase B | | |
|---|---|---|
| Water | qs 100 | g |
| Magnesium sulfate | 0.7 | g |
| Preserving agent (methylparaben) | qs | |

| Phase C | |
|---|---|
| Water | 2 g |
| Preserving agent (diazolinylurea) | qs |

The composition obtained had good cosmetic properties.

### Example 10

### Compacted Powder

A compacted powder comprising the composition below was prepared:

| Composition A: | | |
|---|---|---|
| Talc | 30 | g |
| Bismuth oxychloride | 10 | g |
| Zinc stearate | 4 | g |
| Nylon powder | 20 | g |
| Dispersion of Example 4 | 5 | g |

| Composition B: | |
|---|---|
| Iron oxides | 2 g |
| Liquid petroleum jelly | 6 g |

The powder was obtained in the following manner: composition A was ground in a Kenwood type mill for about 5 minutes with slow stirring, composition B was added and the mixture was ground for about 2 minutes at the same speed, and then for 3 minutes at a faster speed. The preparation was then screened through a 0.16 mm screen, and this mixture was then compacted in compact cases.

A compacted powder that had good cosmetic properties was obtained. The composition obtained was easy and pleasant to apply. It was observed that the film does not migrate into the fine lines of the skin, even after having been worn for several hours.

### Example 11

### Face Gel

The composition below was prepared:

| | | |
|---|---|---|
| Isopropyl palmitate | 10 | g |
| Petroleum jelly (wax) | 5 | g |
| Modified hectorite (clay) | 0.15 | g |
| Ozokerite (wax) | 5 | g |
| Oxyethylenated sorbitan heptaoleate (40 OE) | 5 | g |
| Dispersion of Example 5 | 75 | g |

A gel with good cosmetic properties was obtained.

### Example 12

### Care Oil

The composition below was prepared:

| | |
|---|---|
| Dispersion of Example 2 | 70 g |
| Jojoba oil | 15 g |
| Soybean oil | 15 g |

A care oil that can be applied to the body or the face was obtained.

What is claimed is:

1. A cosmetic composition comprising, in a cosmetically acceptable medium, at least one dispersion of polymer particles in a liquid carbon-based medium, wherein said polymer is chosen from at least one block copolymer comprising at least one first block that is soluble in said liquid carbon-based medium and at least one second block that is insoluble in said liquid carbon-based medium.

2. A cosmetic composition according to claim 1, wherein the at least one block copolymer has a mass polydispersity index (Ip) of less than or equal to 6.

3. A cosmetic composition according to claim 2, wherein the at least one block copolymer has a mass polydispersity index (Ip) ranging from 1.15 to 2.5.

4. A cosmetic composition according to claim 1, wherein the at least one first block and the at least one second block are each chosen from homopolymers and gradient polymers.

5. A cosmetic composition according to claim 1, wherein the at least one block copolymer is chosen from diblock, triblock and multiblock polymers.

6. A cosmetic composition according to claim 1, wherein the at least one block copolymer is chosen from linear polymers.

7. A cosmetic composition according to claim 1, wherein said polymer particles range from 5 nm to 1000 nm in size.

8. A cosmetic composition according to claim 7, wherein said polymer particles range from 30 nm to 200 nm in size.

9. A cosmetic composition according to claim 1, wherein the at least one block copolymer has a number-average molecular weight (Mn) ranging from 1,000 to 700,000.

10. A cosmetic composition according to claim 9, wherein the at least one block copolymer has a number-average molecular weight (Mn) ranging from 25,000 to 150,000.

11. A cosmetic composition according to claim 1, wherein the at least one first block that is soluble in said liquid carbon-based medium comprises from 50% to 100% by weight of at least one monomer that is soluble in said liquid carbon-based medium.

12. A cosmetic composition according to claim 11, wherein the at least one first block that is soluble in said liquid carbon-based medium comprises from 70% to 80% by weight of at least one monomer that is soluble in said liquid carbon-based medium.

13. A cosmetic composition according to claim 1, wherein the at least one second block that is insoluble in said liquid carbon-based medium comprises from 50% to 100% by weight of at least one monomer that is insoluble in said liquid carbon-based medium.

14. A cosmetic composition according to claim 13, wherein the at least one second block that is insoluble in said liquid carbon-based medium comprises from 70% to 80% by weight of at least one monomer that is insoluble in said liquid carbon-based medium.

15. A cosmetic composition according to claim 1, wherein the at least one second block that is insoluble in said liquid carbon-based medium is present in an amount ranging from 30% to 97% by weight, relative to the total weight of the copolymer; and the at least one first block that is soluble in said liquid carbon-based medium is present in an amount ranging from 3% to 70% by weight, relative to the total weight of the copolymer.

16. A cosmetic composition according to claim 15, wherein the at least one second block that is insoluble in said liquid carbon-based medium is present in an amount ranging from 75% to 90% by weight, relative to the total weight of the copolymer.

17. A cosmetic composition according to claim 15, wherein the at least one first block that is soluble in said liquid carbon-based medium is present in an amount ranging from 10% to 25% by weight, relative to the total weight of the copolymer.

18. A cosmetic composition according to claim 1, wherein the at least one block copolymer comprises at least one soluble monomer chosen from at least one of the following monomers:
the methacrylates of formula CH₂═C(CH₃)—COOR₁
wherein R₁ is chosen from linear and branched C8-C22 alkyl groups; cyclic alkyl groups comprising from 8 to 30 carbon atoms; and tert-butyl groups;
the acrylates of formula CH₂═CH—COOR₂
wherein R₂ is chosen from linear and branched C8-C22 alkyl groups; cyclic alkyl groups comprising from 8 to 30 carbon atoms; and isobutyl groups;
the (meth)acrylamides of formulae CH₂═C(CH₃)—CONR₃R₄ and CH₂═CH—CONR₃R₄,
wherein R₃ is chosen from a hydrogen atom and linear and branched C1-C12 alkyl groups and R₄ is chosen from linear and branched C₈ to C₁₂ alkyl groups;
the di-n-alkylitaconates of formula CH₂═C(CH₂—COO(CH₂)ₙ₋₁—CH₃)—COO(CH₂)ₙ₋₁—CH₃,
wherein n is chosen from integers greater than or equal to 5;
the vinyl esters of formula R₅—CO—O—CH═CH₂ wherein R₅ is chosen from linear and branched C₈ to C₂₂ alkyl groups;
the ethers of vinyl alcohol and of an alcohol of formula R₆O—CH═CH₂ wherein R₆ is chosen from linear and branched alkyl groups comprising from 8 to 22 carbon atoms;
ethylenic monomers wherein the ester group contains silanes or siloxanes, and which contain only one silicon atom; and
carbon-based macromonomers with a polymerizable end group.

19. A cosmetic composition according to claim 18 wherein the carbon-based macromonomers with a polymerizable end group are chosen from:
(i) linear and branched C6-C22 alkyl (meth)acrylate homopolymers and copolymers, comprising a polymerizable end group chosen from vinyl and (meth)acrylate groups; and
(ii) polyolefins with an ethylenically unsaturated end group.

20. A cosmetic composition according to claim 19, wherein the polymerizable end group chosen from vinyl and (meth)acrylate groups is chosen from poly(2-ethylhexyl acrylate)macromonomers comprising mono(meth)acrylate end groups; poly(dodecyl acrylate); poly(dodecyl methacrylate)macromonomers comprising mono(meth)acrylate end groups poly(stearyl acrylate); and poly(stearyl methacrylate)macromonomers comprising mono(meth)acrylate end groups.

21. A cosmetic composition according to claim 19, where in the polyolefins with an ethylenically unsaturated end group are chosen from: polyethylene macromonomers, polypropylene macromonomers, macromonomers of polyethylene/polypropylene copolymer, macromonomers of polyethylene/polybutylene copolymer, polyisobutylene macromonomers, polybutadiene macromonomers; polyisoprene macromonomers; polybutadiene macromonomers; and poly(ethylene/butylene)-polyisoprene macromonomers.

22. A cosmetic composition according to claim 11, wherein the at least one first block that is soluble in said liquid carbon-based medium is chosen from:
the methacrylates of formula CH₂═C(CH₃)—COOR₁
wherein R¹ is chosen from linear and branched C8-C22 alkyl groups; cyclic alkyl groups comprising from 8 to 30 carbon atoms; and tert-butyl groups; and
the acrylates of formula CH₂═CH—COOR₂
wherein R₂ is chosen from linear and branched C8-C22 alkyl groups; cyclic alkyl groups comprising from 8 to 30 carbon atoms; and isobutyl groups.

23. A cosmetic composition according to claim 22, wherein the at least one first block that is soluble in said liquid carbon-based medium is chosen from 2-ethylhexyl acrylate, isobornyl(meth)acrylate, lauryl(meth)acrylate, stearyl(meth)acrylate, behenyl(meth)acrylate, isobutyl acrylate and tert-butyl methacrylate.

24. A cosmetic composition according to claim 1, wherein the at least one second block that is insoluble in said liquid carbon-based medium comprises at least one insoluble monomer chosen from the following monomers, and the salts and mixtures thereof:
(i) the (meth)acrylates of formulae: CH₂═C(CH₃)—COOR′₁ and CH₂═CH—COOR′₁
wherein R′₁ is chosen from:
linear and branched alkyl groups comprising from 1 to 6 carbon atoms, optionally comprising in its chain at least one hetero atom chosen from O, N and S; and optionally comprising at least one substituent chosen from —OH, halogen atoms and —NR′R″ groups wherein R′ and R″, which may be identical or different, are each chosen from linear and branched C1-C4 alkyls; optionally substituted with at least one polyoxyalkylene group comprising from 5 to 30 oxyalkylene units; wherein tert-butyl methacrylate and isobutyl acrylate are excluded from this definition;
cyclic alkyl groups comprising from 3 to 6 carbon atoms, optionally comprising in its chain at least one heteroatom chosen from O, N and S; optionally comprising at least one substituent chosen from OH and halogen atoms;
(ii) the (meth)acrylamides of formulae:
CH₂═C(CH₃)—CONR′₃R′₄ and CH₂═CH—CONR′₃R′₄,
wherein:
R′3 and R′4, which may be identical or different, are each chosen from hydrogen atoms and linear and branched alkyl groups comprising from 1 to 6 carbon atoms, optionally comprising at least one substituent chosen from —OH, halogen atoms and —NR′R″ groups wherein R′ and R″, which may be identical or different, are each chosen from linear and branched C1-C4 alkyls; or, alternatively,
R′3 is chosen from a hydrogen atom and R′4 is chosen from a 1,1-dimethyl-3-oxobutyl group;
(iii) ethylenically unsaturated monomers comprising at least one functional group chosen from carboxylic, phosphoric and sulfonic groups and salts thereof;
(iv) the vinyl esters of formula: R′6-COO—CH═CH₂ wherein R′6 is chosen from linear and branched alkyl groups comprising from 1 to 6 atoms, cyclic alkyl groups comprising from 3 to 6 carbon atoms, and aromatic groups;
(v) ethylenically unsaturated monomers comprising at least one tertiary amine functional group;
(vi) styrene and derivatives thereof;
(vii) the di-n-alkylitaconates of formula:
CH₂═C(CH₂—COO(CH₂)ₙ₋₁—CH₃)—COO(CH₂)ₙ₋₁—CH₃, wherein n is an integer ranging from 0 to 4;
(viii) ethylenic monomers wherein the ester group contains silanes, silsesquioxanes, siloxanes or carbosiloxane dendrimers, with the exception of monomers comprising only one silicon atom;
(ix) polydimethylsiloxanes macromonomers; and
(x) oligopeptides functionalized with a (meth)acrylate functional group.

25. A cosmetic composition according to claim 24, wherein the polydimethylsiloxanes macromonomers are chosen from those of formula: 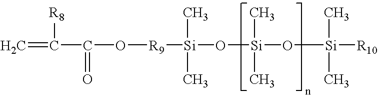
wherein:
R8 is chosen from a hydrogen atom and methyl groups;
R9 is chosen from linear and branched divalent hydrocarbon-based groups comprising from 1 to 10 carbon atoms and optionally comprising one or two ether bonds —O—;
R10 is chosen from linear and branched alkyl groups comprising from 1 to 10 carbon atoms; and
n is chosen from an integer ranging from 1 to 300.

26. A cosmetic composition according to claim 24, wherein the at least one second block that is insoluble in said liquid carbon-based medium comprises at least one insoluble monomer chosen from:
the (meth)acrylates of formulae: CH₂═C(CH₃)—COOR′₁ and CH₂═CH—COOR′₁, wherein R′₁ is as defined in claim 24;
ethylenically unsaturated monomers comprising at least one carboxylic acid functional group and salts thereof;
maleic anhydride;
ethylenic monomers wherein the ester group contains silanes;
polydimethylsiloxanes comprising a monoacryloyloxy or monomethacryloyloxy end group, having the following formula: 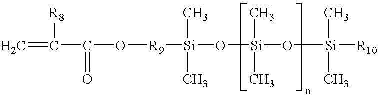
wherein:
R8 is chosen from a hydrogen atom and methyl groups;
R9 is chosen from linear and branched divalent hydrocarbon-based groups comprising from 1 to 10 carbon atoms and optionally comprising one or two ether bonds —O—;
R10 is chosen from linear and branched alkyl groups comprising from 1 to 10 carbon atoms; and
n is an integer ranging from 1 to 300.

27. A cosmetic composition according to claim 26, wherein the at least one second block that is insoluble in said liquid carbon-based medium comprises at least one insoluble monomer chosen from methyl(meth)acrylate, ethyl(meth)acrylate, (meth)acrylic acid, maleic anhydride, (meth)acryloxypropyltris(trimethylsiloxy)silane, (meth)acryloxypropylbis(trimethylsiloxy)methylsilane, (meth)acryloxymethyltris(trimethylsiloxy)silane and (meth)acryloxymethylbis(trimethylsiloxy)methylsilane.

28. A cosmetic composition according to claim 1, wherein the liquid carbon-based medium comprises at least 50% by weight, relative to the total weight of the carbon-based medium, of at least one carbon-based compound that is liquid at 25° C., having a global solubility parameter according to the Hansen solubility space of less than or equal to 20 (MPa)^{1/2}.

29. A cosmetic composition according to claim 28, wherein the liquid carbon-based medium comprises from 70% to 90% by weight, relative to the total weight of the carbon-based medium, of at least one carbon-based compound that is liquid at 25° C., having a global solubility parameter according to the Hansen solubility space of less than or equal to 20 (MPa)^{1/2}.

30. A cosmetic composition according to claim 29, wherein the at least one carbon-based compound is chosen from:
plant oils formed by fatty acid esters of polyols;
linear, branched and cyclic esters, comprising more than 6 carbon atoms;
hydrocarbons;
ketones comprising more than 6 carbon atoms;
aliphatic fatty monoalcohols comprising 6 to 30 carbon atoms, the hydrocarbon-based chain not comprising any substitution groups; and
polyols.

31. A cosmetic composition according to claim 30, wherein the linear, branched and cyclic esters, comprising more than 6 carbon atoms are chosen from RCOOR′ wherein R is chosen from a higher fatty acid residue comprising from 7 to 19 carbon atoms and R′ is chosen from a hydrocarbon-based chain comprising from 3 to 20 carbon atoms.

32. A cosmetic composition according to claim 30, wherein the at least one carbon-based compound is chosen from:
plant oils formed by fatty acid esters of polyols;
the esters of formula RCOOR′ wherein R is chosen from higher fatty acid residues comprising from 7 to 19 carbon atoms and R′ is chosen from hydrocarbon-based chains comprising from 3 to 20 carbon atoms;
volatile and non-volatile, linear and branched C8-C60 alkanes;
volatile and non-volatile, non-aromatic cyclic C5-C12 alkanes;
ethers comprising 7 to 30 carbon atoms;
ketones comprising 8 to 30 carbon atoms; and
aliphatic fatty monoalcohols comprising 12 to 30 carbon atoms, the hydrocarbon-based chain not comprising any substitution groups.

33. A cosmetic composition according to claim 32, wherein the at least one carbon-based compound is chosen from isopropyl myristate, octyidodecanol, C5-C60 isoparaffins, isohexadecane and isononyl isononanoate.

34. A cosmetic composition according to claim 1, wherein said polymer particles in said at least one dispersion are chosen from:
poly(2-ethylhexyl acrylate)-b-poly(methyl acrylate),
poly(isobornyl acrylate)-b-poly(methyl acrylate),
poly(2-ethylhexyl acrylate-co-isobornyl acrylate)-b-poly(methyl acrylate),
poly(2-ethylhexyl acrylate)-b-poly(methyl acrylate-co-acrylic acid),
poly(2-ethylhexyl acrylate-co-acrylic acid)-b-poly(methyl acrylate),
poly(2-ethylhexyl acrylate-co-isobornyl acrylate)-b-poly(methyl acrylate-co-acrylic acid),
poly(2-ethylhexyl acrylate-co-isobornyl acrylate-co-acrylic acid)-b-poly(methyl acrylate),
poly(isobornyl acrylate)-b-poly(methyl acrylate-co-acrylic acid),
poly(isobornyl acrylate-co-acrylic acid)-b-poly(methyl acrylate),
poly(isobutyl acrylate)-b-poly(methyl acrylate),
poly(isobutyl acrylate)-b-poly(methyl acrylate-co-acrylic acid),
poly(isobutyl acrylate-co-acrylic acid)-b-poly(methyl acrylate),
poly(isobutyl acrylate-co-isobornyl acrylate)-b-poly(methyl acrylate),
poly(isobutyl acrylate-co-isobornyl acrylate)-b-poly(methyl acrylate-co-acrylic acid),
poly(isobutyl acrylate-co-isobornyl acrylate-co acrylic acid)-b-poly(methyl acrylate),
poly(2-ethylhexyl acrylate)-b-poly(methyl acrylate)-b-poly(2-ethylhexyl acrylate),
poly(2-ethylhexyl acrylate-co-isobornyl acrylate)-b-poly(methyl acrylate)-b-poly(2-ethylhexyl acrylate-co-isobornyl acrylate),
poly(2-ethylhexyl acrylate-co-acrylic acid)-b-poly(methyl acrylate)-b-poly(2-ethylhexyl acrylate-co-acrylic acid),
poly(2-ethylhexyl acrylate)-b-poly(methyl acrylate-co-acrylic acid)-b-poly(2-ethylhexyl acrylate),
poly(2-ethylhexyl acrylate-co-isobornyl acrylate-co-acrylic acid)-b-poly(methyl acrylate)-b-poly(2-ethylhexyl acrylate-co-isobornyl acrylate-co-acrylic acid), and
poly(2-ethylhexyl acrylate-co-isobornyl acrylate)-b-poly(methyl acrylate-co-acrylic acid)-b-poly(2-ethylhexyl acrylate-co-isobornyl acrylate).

35. A cosmetic composition according to claim 34, wherein the at least one dispersion of polymer particles is in an alkane.

36. A cosmetic composition according to claim 35, wherein the at least one dispersion of polymer particles is in isododecane.

37. A cosmetic composition according to claim 1, wherein said at least one dispersion has a solids content ranging from 5% to 80% by weight, relative to the total weight of the composition.

38. A cosmetic composition according to claim 37, wherein said at least one dispersion has a solids content ranging from 18% to 25% by weight, relative to the total weight of the composition.

39. A cosmetic composition according to claim 1, wherein the at least one dispersion is present in an amount ranging from 0.1% to 90% by weight, relative to the total weight of the composition.

40. A cosmetic composition according to claim 39, wherein the at least one dispersion is present in an amount ranging from 5% to 70% by weight, relative to the total weight of the composition.

41. A cosmetic composition according to claim 1, further comprising at least one component chosen from fatty phases, hydrophilic phases, dyestuffs, polymers, vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides.

42. A cosmetic composition according to claim 1, wherein the composition is in the form of a makeup composition.

43. A cosmetic composition according to claim 42, wherein the makeup composition is in a form chosen from: a complexion product; a lip product; a concealer product; a blusher, a mascara; an eyeliner; an eyebrow makeup product, a lip pencil; an eye pencil; a nail product; a body makeup product; a hair makeup product; a composition for protecting or caring for the skin of the face, the neck, the hands or the body; an anti-sun composition; a self-tanning composition; and a hair product.

44. A dispersion of polymer particles in a liquid carbon-based medium, wherein said polymer particles are chosen from at least one block copolymer comprising at least one first block that is soluble in said liquid carbon-based medium and at least one second block that is insoluble in said liquid carbon-based medium,
wherein the liquid carbon-based medium comprises at least one carbon-based compound chosen from:
plant oils formed by fatty acid esters of polyols;
the esters of formula RCOOR′ wherein R is chosen from higher fatty acid residues comprising 7 to 19 carbon atoms and R′ is chosen from hydrocarbon-based chains comprising from 3 to 20 carbon atoms;
volatile and non-volatile, linear and branched C8-C60 alkanes;
volatile and non-volatile, non-aromatic, cyclic C5-C12 alkanes;
ethers comprising 7 to 30 carbon atoms;
ketones comprising 8 to 30 carbon atoms; and
aliphatic fatty monoalcohols comprising 12 to 30 carbon atoms, the hydrocarbon-based chain not comprising any substitution groups.

45. A cosmetic process for making up, cleansing, protecting against the sun, shaping, dyeing or caring for keratin materials comprising applying to said keratin materials at least one cosmetic composition comprising, in a cosmetically acceptable medium, at least one dispersion of polymer particles in a liquid carbon-based medium, wherein said polymer is chosen from at least one block copolymer comprising at least one first block that is soluble in said liquid carbon-based medium and at least one second block that is insoluble in said liquid carbon-based medium.

46. A cosmetic process according to claim 45, wherein said keratin materials are chosen from body or facial skin, the nails, the hair and the eyelashes.
